SEQUENCE LISTING (35 sequences)

SEQ ID NO: 1 - DNA, 50 bp, Homo sapiens
gagaccctgactctacgaaaattaaaagttagcccggtgtggtggcgcgc

SEQ ID NO: 2 - DNA, 50 bp, Homo sapiens
cgcttgcatgctcacctctggcagcagggcagtcacggctccgccatgga

SEQ ID NO: 3 - DNA, 50 bp, Homo sapiens
ggcaggatgattgcttaagcccaggagttcggggttacagtgagctgtgg

SEQ ID NO: 4 - DNA, 50 bp, Homo sapiens
gagggcaagccaccaaattacctaggctgaggttagagagattggccagc

SEQ ID NO: 5 - DNA, 50 bp, Homo sapiens
gagctcgcctatgagcaagtggcaaaggccctcaaataagcccctcctgg

SEQ ID NO: 6 - DNA, 50 bp, Homo sapiens
atactggaggggcagtgggtgccagtagtgtcctgctggtccttcctgaa

SEQ ID NO: 7 - DNA, 50 bp, Homo sapiens
ccatggtgatggatggtttggaaagggaatgttggtgccttttgtgccac

SEQ ID NO: 8 - DNA, 50 bp, Homo sapiens
tgtggtgaagaggcagaaccgaggtcgggagcccattgatgtctgagcct

SEQ ID NO: 9 - DNA, 50 bp, Homo sapiens
ccccagtgtgtataagctggcatttcgccagcttgtacgtagcttgccac

SEQ ID NO: 10 - DNA, 50 bp, Homo sapiens
gggaagagagtttgcaggcctgtgcttcaaagaactcttctagtctgctt

SEQ ID NO: 11 - DNA, 50 bp, Homo sapiens
gcccttcaccattgtggaatgatgccctggctttaaggtttagctccaca

SEQ ID NO: 12 - DNA, 50 bp, Homo sapiens
tgagatctacaagttgacccgcaaggaggcccaggagtaggaggctgcag

SEQ ID NO: 13 - DNA, 50 bp, Homo sapiens
gaagggtccccctgctgactggagagctgggaatatggcatttggacact

SEQ ID NO: 14 - DNA, 50 bp, Homo sapiens
gacagatttgttcatagaccctctcaagtgccctctccgagctggtaggc

SEQ ID NO: 15 - DNA, 50 bp, Homo sapiens
gcctcttctctcaagcctgcttcagatcataagttcttccacacatctcc

SEQ ID NO: 16 - DNA, 50 bp, Homo sapiens
gtgccgagctgtccatagctgcagtgaaaggtgaagagcaagaccttctc

SEQ ID NO: 17 - DNA, 50 bp, Homo sapiens
gcttctccagatccccagcgccaggagtggtttgcccggtacttcacatt

SEQ ID NO: 18 - DNA, 50 bp, Homo sapiens
cctttattttgctgcttgatgatgagagggagggctgctgccacagactg

SEQ ID NO: 19 - DNA, 50 bp, Homo sapiens
atggagctgtctttcagatctttcctgggattacccctgcctacccccag

SEQ ID NO: 20 - DNA, 50 bp, Homo sapiens
tgcagccgtccatagcagtacccctaaaatcccaccagaatacgggtccc

SEQ ID NO: 21 - DNA, 50 bp, Homo sapiens
caccagaagtagcagagaagcagggggccagagctacaacagtattcttc

SEQ ID NO: 22 - DNA, 50 bp, Homo sapiens
acttctacttgccaacatctgccttgctggacttgtatgggattgtctcc

SEQ ID NO: 23 - DNA, 50 bp, Homo sapiens
agtgaccattgaaaatcaccctcatctgccaccacctcccatgtgttcag

SEQ ID NO: 24 - DNA, 50 bp, Homo sapiens
ttgtcatcctttgagctgctgactgccaataacattcttcgatctctggg

SEQ ID NO: 25 - DNA, 50 bp, Homo sapiens
gtcgctcggacccaggacactacagtttctctatgcgatctccagagctc

SEQ ID NO: 26 - DNA, 50 bp, Homo sapiens
accgggacatccggctgatggtcatggagatccgcaatgcttatgctgtg

SEQ ID NO: 27 - DNA, 50 bp, Homo sapiens
ggacattaagttcagtggccatgttagctgggtcgggaagacatccatgg

SEQ ID NO: 28 - DNA, 50 bp, Homo sapiens
tggaagatcagacccagctccttacccttgtctgccagttgtaccagggc

SEQ ID NO: 29 - DNA, 50 bp, Homo sapiens; identical to SEQ ID NO: 6

SEQ ID NO: 30 - DNA, 50 bp, Homo sapiens; identical to SEQ ID NO: 7

SEQ ID NO: 31 - DNA, 50 bp, Homo sapiens
ggtccccatgtgcctgttgttcagccctctctcttgttccctttctgagc

SEQ ID NO: 32 - DNA, 50 bp, Homo sapiens
gaaccaggagacacgtgtggcgggcagtgggacccatagatcttctgaaa

SEQ ID NO: 33 - DNA, 50 bp, Homo sapiens
gtaacggagtttagagccagggctgatgctttggtgtggccagcactctg

SEQ ID NO: 34 - DNA, 50 bp, Homo sapiens
aaggctcgatcctgcgagaaggaagtggtctctgcccagcctgccacctt

SEQ ID NO: 35 - DNA, 50 bp, Homo sapiens
cctcaacaggcccagggagggaagtgtgagcgccttggtatgacttaaaa

(19) United States
(12) Patent Application Publication
(10) Pub. No.: US 2015/0197806 A1
(43) Pub. Date: Jul. 16, 2015

(54) BIOMARKERS FOR DETERMINING THE M. TUBERCULOSIS INFECTION STATUS

(71) Applicant: Nottingham Trent University, Nottingham (GB)

(72) Inventor: Graham Roy Ball, Nottingham (GB)

(21) Appl. No.: 14/410,006

(22) PCT Filed: Jun. 21, 2013
(86) PCT No.: PCT/GB2013/051635

§ 371 Date: Dec. 19, 2014

(30) Foreign Application Priority Data
Jun. 22, 2012 (GB) 1211158.9

(51) Int. Cl. C12Q 1/68

(52) U.S. Cl. CPC C12Q 1/6883; C12Q 2600/158; C12Q 2600/112

(57) ABSTRACT

The present invention relates to novel biomarkers for determining the Mycobacterium tuberculosis infection status of a subject, and to uses of novel panels of biomarkers. A method of determining the M. tuberculosis infection status of a subject comprising providing a sample of material obtained from the subject determining the expression level of one or more genes selected from the group consisting of: NXNL1, PSMA7, C6orf61, EMP1, CLIC1, LACTB and DUSP3 in the sample.

The present invention relates to novel biomarkers for determining the Mycobacterium tuberculosis infection status of a subject, and to uses of novel panels of biomarkers.

Tuberculosis (TB) is an infection caused by Mycobacterium tuberculosis (M. tuberculosis) and is a major cause or morbidity and mortality worldwide. M. tuberculosis is an airborne bacterial infection that primarily affects the lungs. It is estimated that approximately 2.2 billion people or a third of the world's population are infected with M. tuberculosis.

The majority of infected people remain asymptomatic. Infected people who remain asymptomatic are said to have latent M. tuberculosis infection (latent TB). A person infected with M. tuberculosis has about a 10% lifetime risk of developing active M. tuberculosis infection (active TB) where symptoms of M. tuberculosis infection are shown.

M. tuberculosis is a substantial management and cost burden for healthcare systems and could be reduced with improvements in diagnosis and informed patient management. Treatments for M. tuberculosis infection vary depending on the type of M. tuberculosis infection status that a person has. A person who has latent M. tuberculosis infection and does not have active M. tuberculosis infection may be given preventative therapy.

Current tests for M. tuberculosis infection can not distinguish between latent and active M. tuberculosis infection and cannot identify which individuals having latent M. tuberculosis infection will go on to develop active M. tuberculosis infection.

It would be advantageous to be able to test for the infection status of an individual subject to distinguish between uninfected individuals and individuals having latent M. tuberculosis infection and active M. tuberculosis infection. Such a test could also be used to follow the infection status of individuals to determine whether TB is being activated or during treatment to determine when the M. tuberculosis infection has been cleared.

In a first aspect the present invention provides a method of determining the M. tuberculosis infection status of a subject comprising:
(a) providing a sample of material obtained from the subject; and
(b) determining the expression level of one or more genes selected from the group consisting of: NXNL1, PSMA7, C6orf61, EMP1, CLIC1, LACTB and DUSP3 in the sample.
Optionally, the method could comprise a step (c) determining the expression level of one or more genes selected from table 3 or table 4.

The method may further comprise: (d) employing the expression level determined in (b) and optionally (c) to distinguish between subjects not infected with M. tuberculosis, subjects with latent M. tuberculosis infection and subjects with active M. tuberculosis infection.

Subjects not infected with M. tuberculosis may be defined as subjects that test negative for M. tuberculosis using a tuberculin-skin test (TST) and/or test negative for M. tuberculosis using an antigen-specific IFN-gamma release assay (IGRA).

Subjects with latent M. tuberculosis infection may be defined as subjects that test positive for M. tuberculosis using a tuberculin-skin test (TST) and/or test positive for M. tuberculosis using an antigen-specific IFN-gamma release assay (IGRA) but do not have symptoms of tuberculosis such as a persistent cough.

Subjects with active M. tuberculosis infection may be confirmed by culture for M. tuberculosis from a blood, serum or sputum sample and also test positive for M. tuberculosis using a tuberculin-skin test (TST) and/or test positive for M. tuberculosis using an antigen-specific IFN-gamma release assay (IGRA) and also have symptoms of tuberculosis.

The sample of material may be a sample of blood, sputum, saliva, wound exudate, urine, faeces, peritoneal fluid or any respiratory secretion. A sample of blood may be whole blood, blood plasma or blood serum.

Preferably the sample is a sample of whole blood. Blood samples have the advantage that they are readily obtainable and tend to be more homogenous in nature than other sample types. Samples of whole blood contain RNA which can be extracted to generate a transcriptional profile.

The expression level of one or more genes selected from the group consisting of: NXNL1, PSMA7, C6orf61, EMP1, CLIC1, LACTB and DUSP3 or selected from table 3 or table 4 in a sample may be determined by any suitable method. Such methods include methods that quantify the nucleotide products of these genes such as: quantitative PCR using suitable oligonucleotide primers designed to adhere within the sequence of an mRNA encoded by the gene of interest; analysis of expression arrays; next generation sequencing, comparative genomic hybridisation arrays (CGH arrays); multiplexed PCR. The expression level of one or more genes selected from the group consisting of: NXNL1, PSMA7, C6orf61, EMP1, CLIC1, LACTB and DUSP3 or selected from table 3 or table 4 in a sample may also be determined using methods that quantify the protein products of the selected genes, for example: ELISA immunohistochemistry; protein aptamer arrays or protein immunological arrays.

The expression level of the gene may be determined by measuring the rate of polymerisation of the RNA using standard techniques.

The method of the invention may not include the step of obtaining the sample.

The method of the invention may include a further step of comparing the determined value of the expression level of one or more genes selected from the group consisting of: NXNL1, PSMA7, C6orf61, EMP1, CLIC1, LACTB and DUSP3 or selected from table 3 or table 4 in a sample with a reference value.

The reference value may be the value for the expression level of the same gene in a sample of the same sample type, from an individual who is known to have or not to have infection with M. tuberculosis. Alternatively, or additionally, the reference value may be the expression level of the same gene in the same sample type in a sample taken previously from the same subject, for example, prior to or during the course of a particular treatment. The reference sample may be a sample of the same type, for example, both samples may be blood samples. In this way the expression level of one or more genes selected from the group consisting of: NXNL1, PSMA7, C6orf61, EMP1, CLIC1, LACTB and DUSP3 or selected from table 3 or table 4 in a sample may be used to monitor the progression of an infection in a subject, and/or to monitor the efficacy of a particular treatment in a subject.

Alternatively or in addition the reference value may be the expression level for the selected gene in an individual that is infected with M. tuberculosis or the reference value may be the expression level for the selected gene in an individual that is not infected with M. tuberculosis.

The method of the invention may be carried out in vitro.

The subject may be a mammal, and is preferably a human, but may alternatively be a monkey, ape, cat, dog, cow, horse, deer, badger, rabbit or rodent.

The method may be used in one or more of the following; diagnosing whether or not a subject has M. tuberculosis infection; advising on the prognosis for a subject with a M. tuberculosis infection; and monitoring the effectiveness or response of a subject to a particular treatment for infection by M. tuberculosis.

In the method altered expression level of one or more genes selected from: NXNL1, PSMA7, C6orf61 and EMP1 and optionally one or more genes selected from table 3 can differentiate between subjects not infected with M. tuberculosis and subjects with latent M. tuberculosis infection. NXNL1, PSMA7, C6orf61 and EMP1 and optionally one or more genes selected from table 3 may be used as a biomarker panel to distinguish between subjects not infected with M. tuberculosis and subjects with latent M. tuberculosis infection with a high degree of accuracy. This biomarker panel is advantageous because it provides a high degree of accuracy with only a small number of biomarkers. The evaluation error of this biomarker panel is 0.93%. Evaluation error is calculated as the square root of the averaged difference between model predictions and the actual TB status squared, expressed as a percentage.

In subjects with latent M. tuberculosis infection the expression levels of NXNL1, PSMA7 and C6orf61 are lower then in subjects not infected with M. tuberculosis. In subjects with latent M. tuberculosis infection the expression levels of EMP1 are higher than in subjects not infected with M. tuberculosis. Therefore, if these four markers are used together as a biomarker panel a pattern of decreased expression of NXNL1, PSMA7 and C6orf61 and also increased expression of EMP1 (compared to the level expected in a control subject not infected with M. tuberculosis) indicates that the subject may have latent M. tuberculosis infection.

In the method altered expression level of one, two, three or all of the genes NXNL1, PSMA7, C6orf61 and EMP1 may be evaluated together to differentiate between subjects not infected with M. tuberculosis and subjects with latent M. tuberculosis infection.

In order to improve the accuracy of the biomarker panel for distinguishing subjects that are not infected with M. tuberculosis and subjects with latent M. tuberculosis infection one, two or three biomarkers listed in table 3 may be tested in addition to NXNL1, PSMA7, C6orf61 and EMP1.

One, two or three of the following biomarkers may be tested in addition to NXNL1, PSMA7, C6orf61 and EMP1 wherein increased expression of LOC389541 and/or increased expression of MID1IP1 and/or increased expression of KLRC3 and/or increased expression of KLF9 and/or decreased expression GPR117 and/or increased expression of FBXO32 and/or decreased expression of TAZ and/or increased expression of C5ORF29 and/or decreased expression of HSDL1 and/or increased expression of CHUK and/or increased expression of LOC652062 and/or decreased expression of HIP1 and/or increased expression of C6ORF60 and/or increased expression of MTMR11 indicates that a subject has latent M. tuberculosis infection.

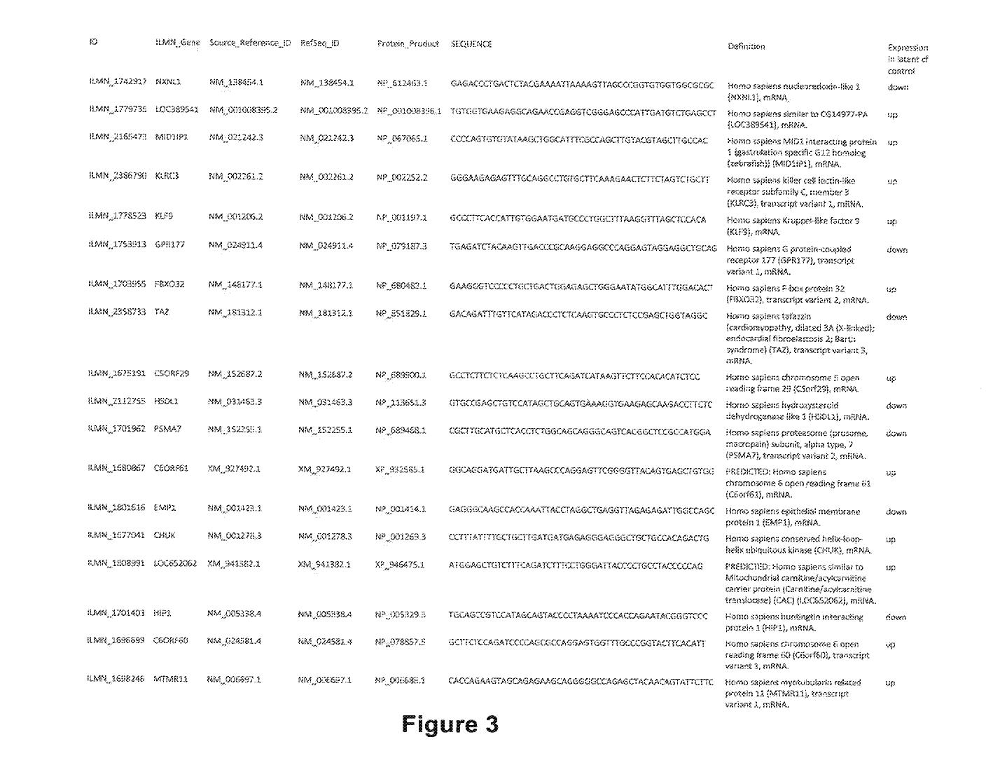

The full names and nucleotide sequences of each of the biomarkers useful in distinguishing between subjects that are not infected with M. tuberculosis and subjects with latent M. tuberculosis infection is shown in FIG. 3. The right hand column of FIG. 3 shows whether the biomarker expression is increased (up) or decreased (down) in subjects that have latent M. tuberculosis infection.

In the method altered expression level of one or more genes selected from: CLIC1, LACTB and DUSP3 can differentiate between subjects with latent M. tuberculosis infection and subjects with active M. tuberculosis infection.

Altered expression level of one, two or all of the genes CLIC1, LACTB and DUSP3 may be evaluated together to differentiate between subjects with latent M. tuberculosis infection and subjects with active M. tuberculosis infection.

In the method altered expression level of one or more genes selected from: CLIC1, LACTB and DUSP3 and optionally one or more genes selected from table 4 can differentiate between subjects with active M. tuberculosis infection and subjects with latent M. tuberculosis infection. CLIC1, LACTB and DUSP3 and optionally one or more genes selected from table 4 may be used as a biomarker panel to distinguish between subjects with active M. tuberculosis infection and subjects with latent M. tuberculosis infection with a high degree of accuracy. This biomarker panel is advantageous because it provides a high degree of accuracy with only a small number of biomarkers. The evaluation error of this biomarker panel is 0.26%.

In subjects with active M. tuberculosis infection the expression levels of CLIC1, LACTB and DUSP3 are higher than in subjects with latent M. tuberculosis infection or subjects not infected with M. tuberculosis. Therefore, if these three markers are used together as a biomarker panel a pattern of increased expression of CLIC1, LACTB and DUSP3 (compared to the level expected in a subject with latent M. tuberculosis infection or subjects not infected with M. tuberculosis) indicates that the subject may have active M. tuberculosis infection.

The sensitivity of the biomarker panel may be increased by additionally testing one or more biomarkers listed in table 4. If the level of any of the biomarkers in table 4 is increased this indicates that the subject may have active M. tuberculosis infection.

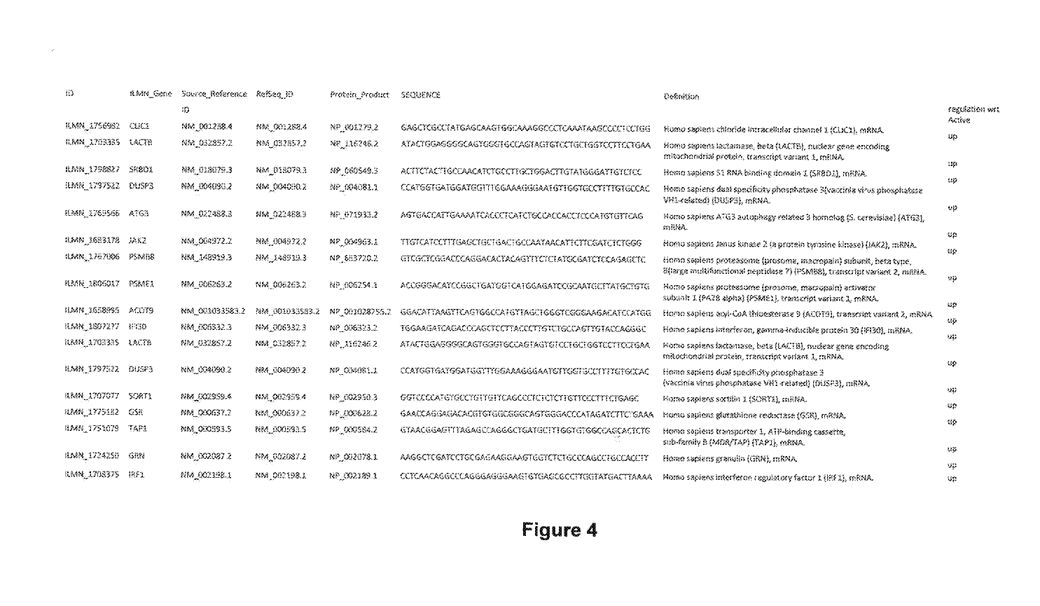

The full names and nucleotide sequences of each of the biomarkers useful for testing whether a subject has active M. tuberculosis infection or latent M. tuberculosis infection is shown in FIG. 4. The right hand column of FIG. 4 shows whether the biomarker expression is increased (up) or decreased (down) in subjects that have active M. tuberculosis infection.

A subject may be tested for expression levels of one, two, three, four, five, six, or all seven biomarkers selected from the group consisting of: NXNL1, PSMA7, C6orf61, EMP1, CLIC1, LACTB and DUSP3. This may allow the M. tuberculosis infection status of the subject to be determined as comparing the expression levels of a number of or all of these biomarkers may allow a subject to be classified as a subject not infected with M. tuberculosis, a subject with active M. tuberculosis infection or a subject with latent M. tuberculosis infection.

The altered expression level is an expression level that is higher or lower than the expression level expected in a subject not infected with M. tuberculosis. Higher or lower expression level means an expression level that is statistically significantly higher or lower than the control expression level that it is compared to. Statistical significance may be measured using standard statistical methods. Higher or lower expression level may be a statistically significantly higher or lower expression level when the significance is corrected using the number of samples.

The expression level of the selected gene may be determined using the level of mRNA encoded by that gene present in the sample.

The expression level of the selected gene may be determined using the level of mRNA encoded by that gene present in the sample.

The expression level of one or more further genes may also be determined.

According to another aspect the invention provides a kit for use in determining the M. tuberculosis infection status in a subject comprising at least one agent for determining the expression level of one or more genes selected from the group consisting of: NXNL1, PSMA7, C6orf61, EMP1, CLIC1, LACTB and DUSP3 in a sample from a subject and instructions for determining the M. tuberculosis infection status of the subject. The instructions may include instructions to perform an assay for expression level of one or more of the selected genes. The instructions may provide reference values for comparison with values for the expression level of the selected gene.

The agent may an oligonucleotide, for example an oligonucleotide that adheres to a mRNA encoded by the selected gene. The kit may provide a pair of oligonucleotides suitable for amplifying the selected gene or an mRNA thereof.

The expression level of one or more genes selected from the group consisting of: NXNL1, PSMA7, C6orf61, EMP1, CLIC1, LACTB and DUSP3 may be used as a means to determine the M. tuberculosis infection status in a subject.

In another aspect the present invention provides a gene expression product from a gene selected from the group consisting of NXNL1, PSMA7, C6orf61, EMP1, CLIC1, LACTB and DUSP3 or a gene selected from table 3 or table 4 for use as biomarker for infection by M. tuberculosis.

In another aspect the present invention provides an oligonucleotide capable of detecting the presence or expression level of a gene expression product from a gene selected from the group consisting of NXNL1, PSMA7, C6orf61, EMP1, CLIC1, LACTB and DUSP3 or a gene selected from table 3 or table 4 in a sample from a subject.

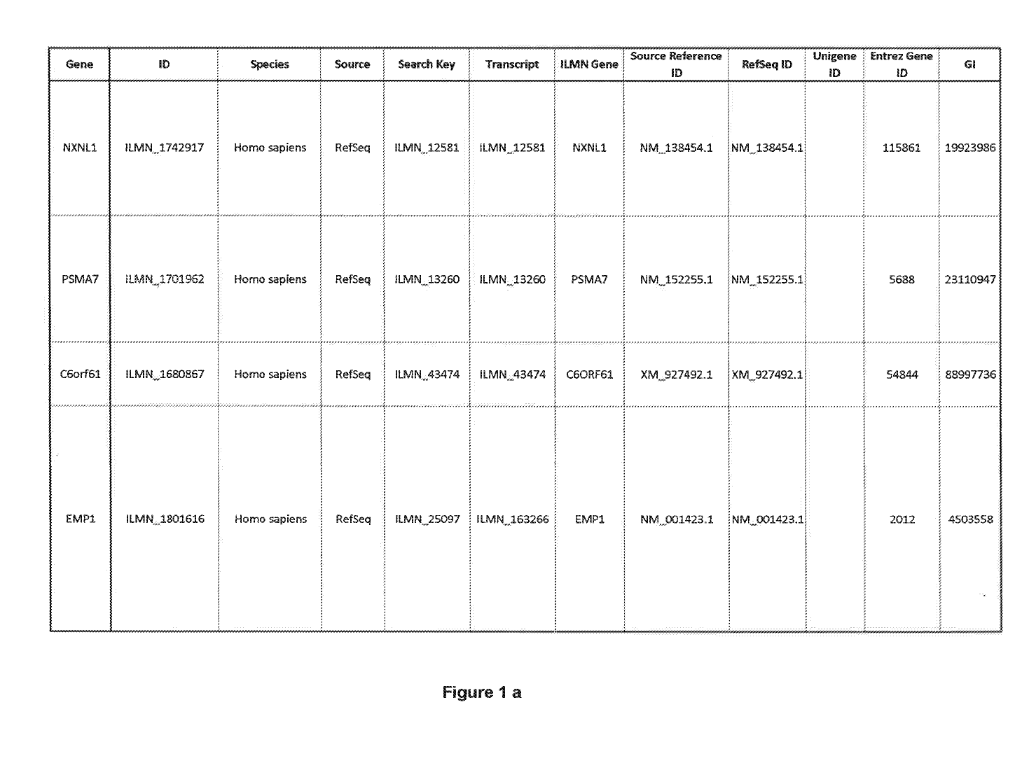

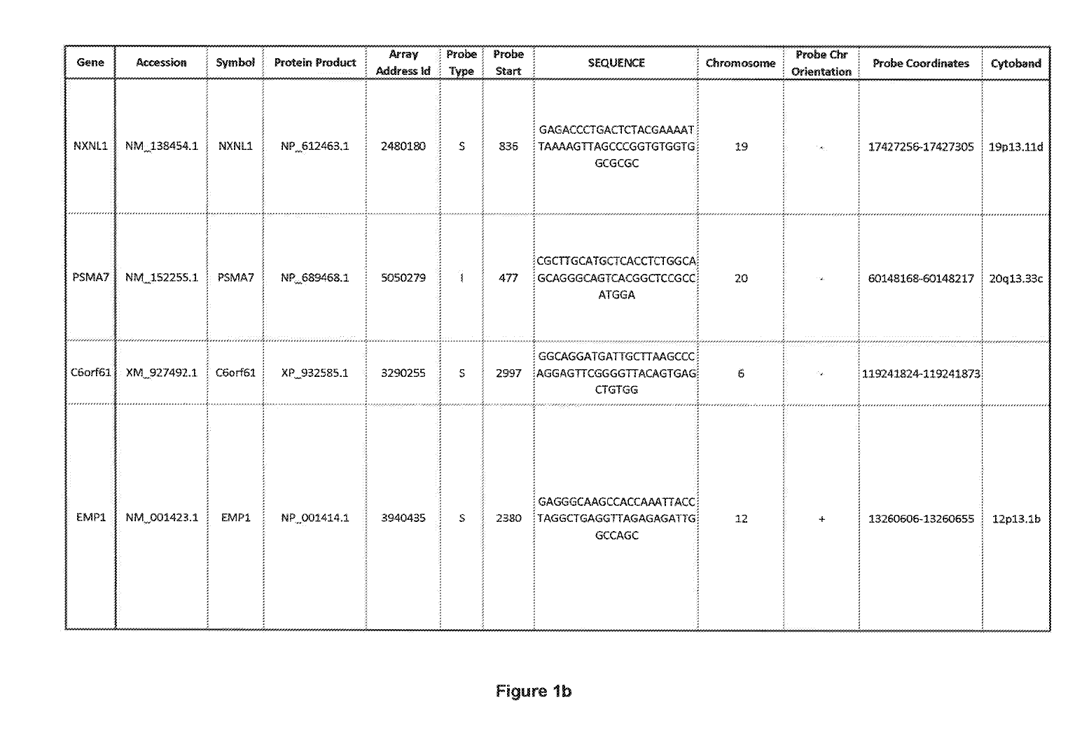

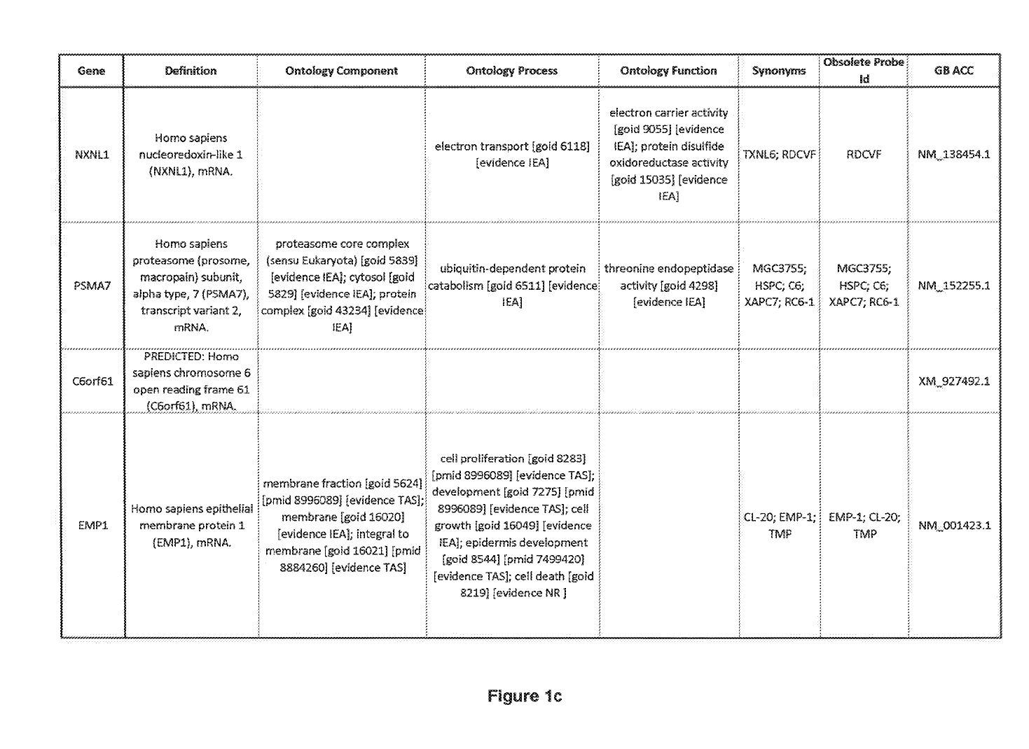

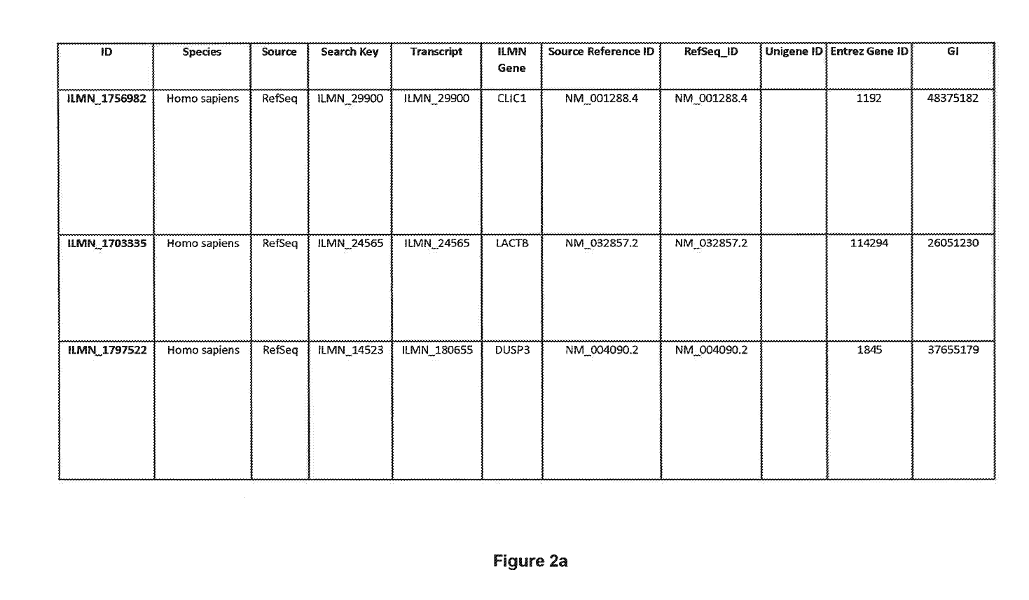

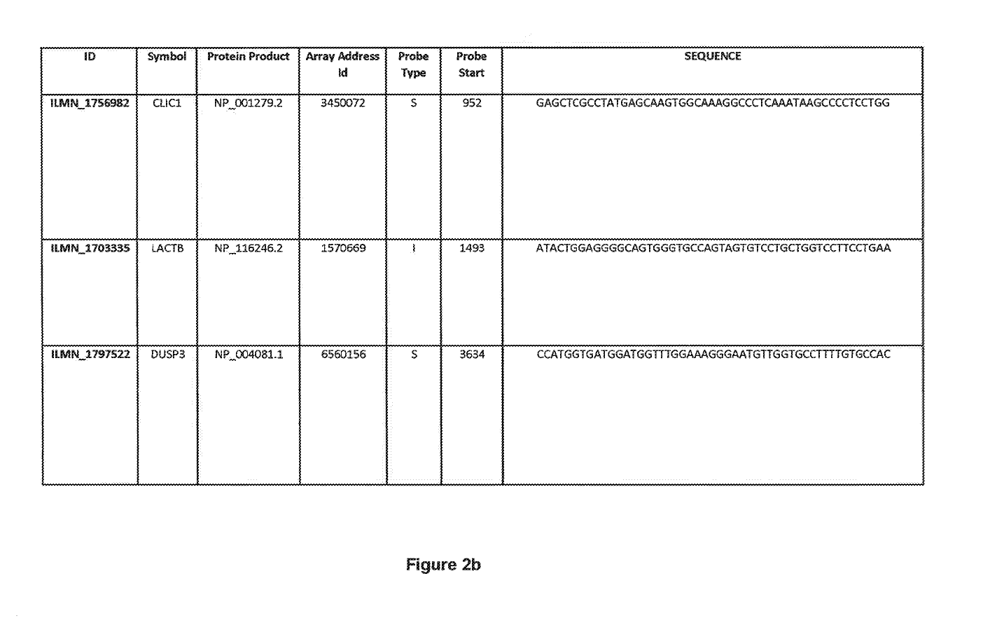

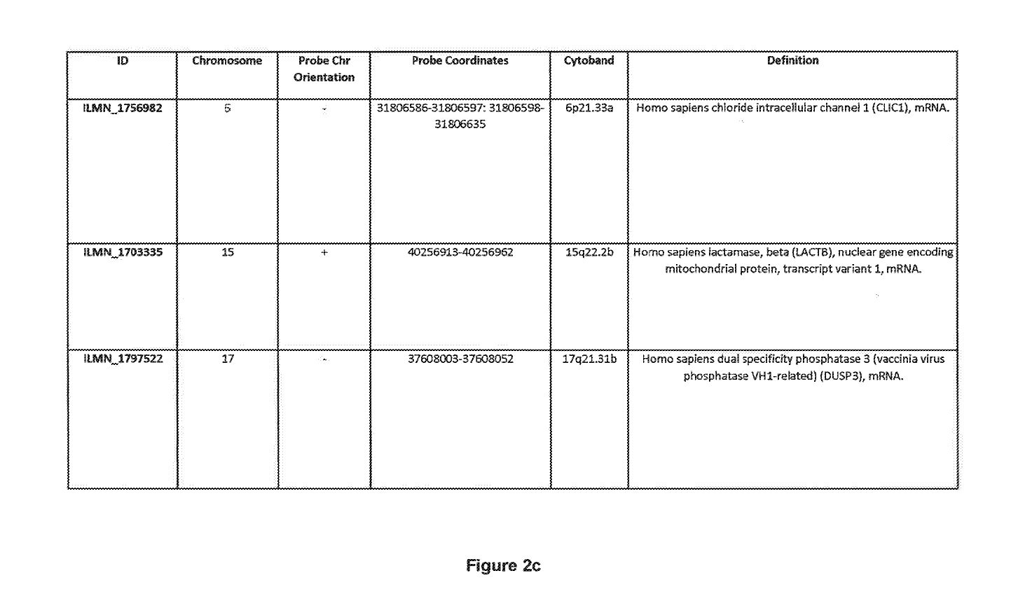

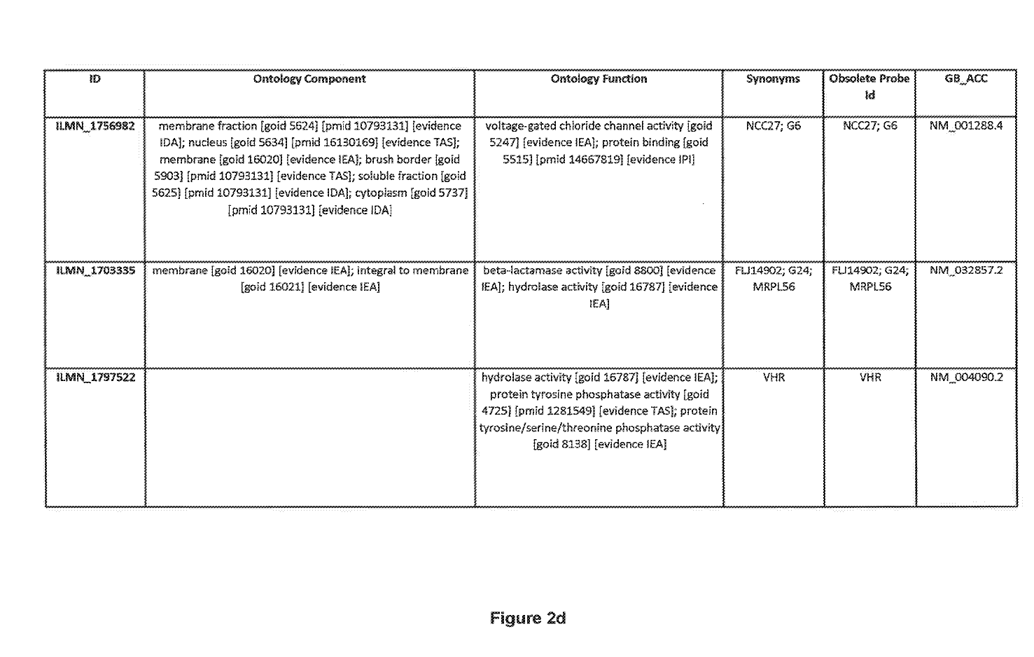

The nucleotide sequences shown for each gene in FIG. 1 and FIG. 2, FIG. 3 and FIG. 4 are sections of the nucleotide sequence for each gene that are particularly useful as representative sequences for diagnosis. Therefore it is particularly advantageous to design a pair of primers for identification of each gene that would bind within the sequence given for that gene in FIG. 1, FIG. 2, FIG. 3 or FIG. 4. The primers may be designed to amplify part of all of the given nucleotide sequence.

In another aspect the present invention provides a method, kit, use, gene or oligonucleotide as described herein with reference to the examples.

The use of an altered expression level of one or more genes selected from: CLIC1, LACTB and DUSP3 to differentiate between subjects with active M. tuberculosis infection and subjects with latent M. tuberculosis infection or the use of an altered expression level of one or more genes selected from: NXNL1, PSMA7, C6orf61 and EMP1 to differentiate between subjects not infected with M. tuberculosis and subjects with latent M. tuberculosis infection has many applications positively impacting on clinical care. It permits both the regular screening of susceptible populations and also the testing of all individuals who are suspected of M. tuberculosis infection, and thus enables more timely eradication treatment for initial infection, possibly preventing chronic infection. It also permits a quantitative assessment of the efficacy of antibiotic therapy resulting in interventions that are customised to the response of the individual patient. It also provides a tool for widespread use in epidemiological studies, an important consideration M. tuberculosis infection becomes more prevalent.

NXNL1, PSMA7, C6orf61, EMP1, CLIC1, LACTB and DUSP3 and a gene selected from table 3 or table 4 may be targets for development of new therapeutics for treating M. tuberculosis infection.

The skilled man will appreciate that preferred features of any one embodiment and/or aspect of the invention may be applied to all other embodiments or aspects of the invention.

The present invention will be further described in more detail, by way of example only, with reference to the following figures in which:

FIG. 1—shows a table of information about the panel of biomarkers that are useful in differentiating between subjects that are not infected with M. tuberculosis and subjects with latent M. tuberculosis infection, these genes are: Homo sapiens nucleoredoxin-like 1 (NXNL1); Homo sapiens proteasome (prosome, macropain) subunit, alpha type, 7 (PSMA7); Homo sapiens chromosome 6 open reading frame 61 (C6orf61); and Homo sapiens epithelial membrane protein 1 (EMP1). The RNA sequences shown are mRNA sequences.

FIG. 2 shows a table of information about the panel of biomarkers that are useful in differentiating between subjects that have active M. tuberculosis infection and subjects with latent M. tuberculosis infection, these genes are: homo sapiens chloride intracellular channel 1 (CLIC1); Homo sapiens lactamase, beta (LACTB, nuclear gene encoding mitochondrial protein, transcript variant 1; and Homo sapiens dual specificity phosphatise 3 (vaccinia virus phosphatise VH1-related)(DUSP3). The RNA sequences shown are mRNA sequences.

FIG. 3—shows a table of information about the panel of additional biomarkers that are useful in differentiating between subjects that are not infected with M. tuberculosis and subjects with latent M. tuberculosis infection, column 6 shows a sequence that is useful in identifying the gene and column 8 shows whether the gene is up or down regulated in subjects with latent M. tuberculosis infection.

FIG. 4—shows a table of information about the panel of biomarkers that are useful in differentiating between subjects that have active M. tuberculosis infection and subjects with latent M. tuberculosis infection, column 6 shows a sequence that is useful in identifying the gene and column 8 shows whether the gene is up or down regulated in subjects with active M. tuberculosis infection.

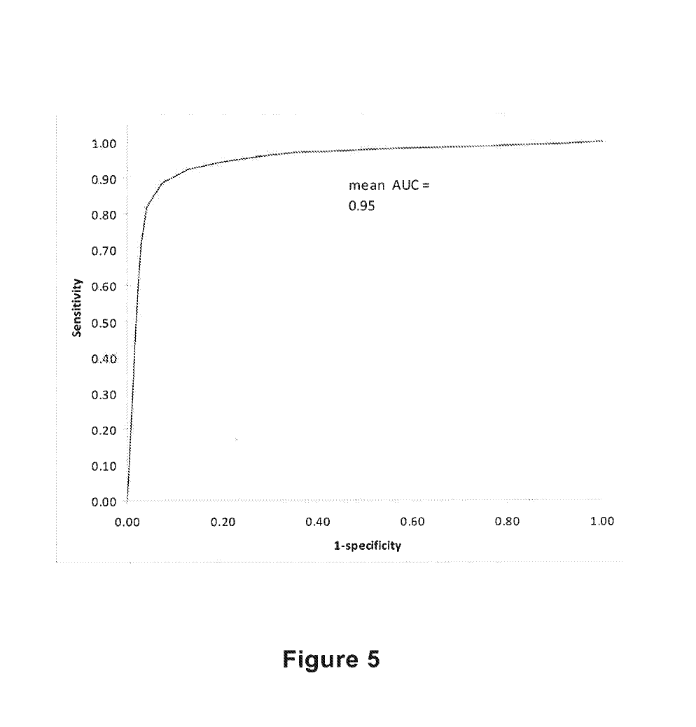

FIG. 5—shows a ROC curve for the core set of biomarkers to distinguish between subjects that are not infected with M. tuberculosis and subjects with latent M. tuberculosis infection (NXNL1, PSMA7, C6orf61 and EMP1), ROC curves show the performance of the classifier incorporating both sensitivity and specificity. The higher the area under the ROC curve the better the performance of the classifier.

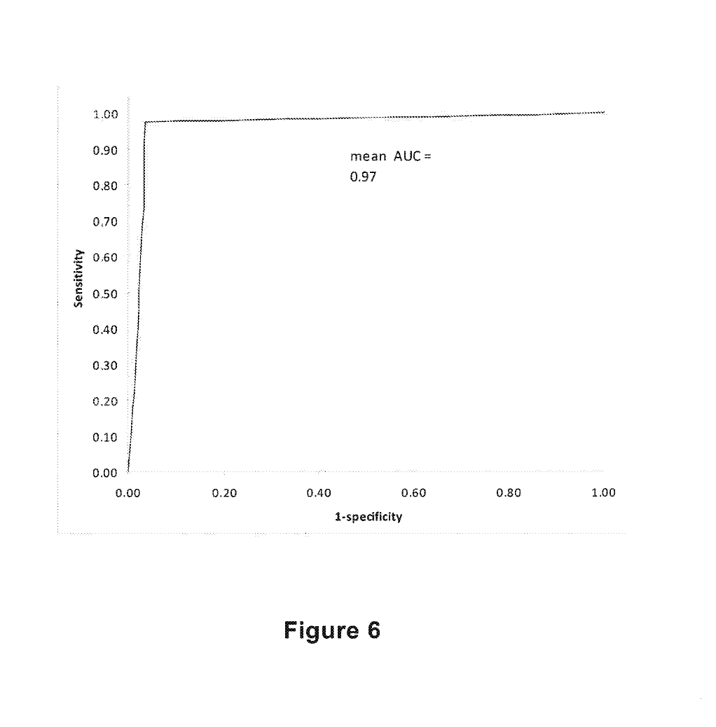

FIG. 6—shows a ROC curve for the core set of biomarkers to distinguish between subjects that have active M. tuberculosis infection and subjects with latent M. tuberculosis infection (CLIC1, LACTB and DUSP3), ROC curves show the performance of the classifier incorporating both sensitivity and specificity. The higher the area under the ROC curve the better the performance of the classifier.

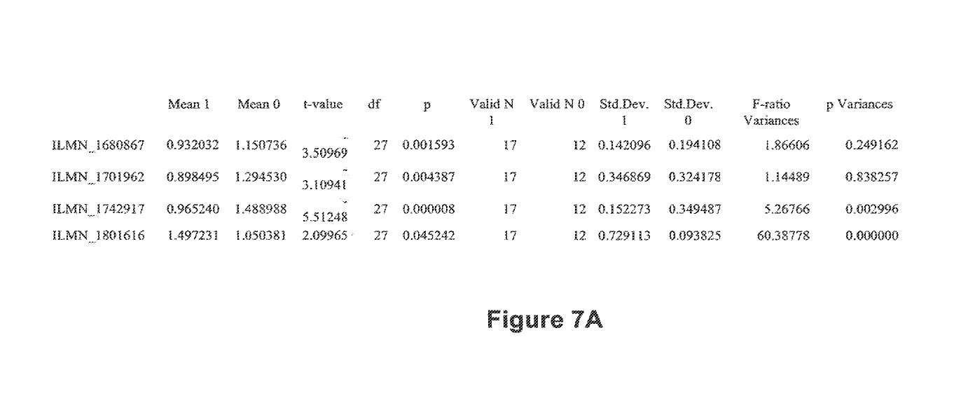

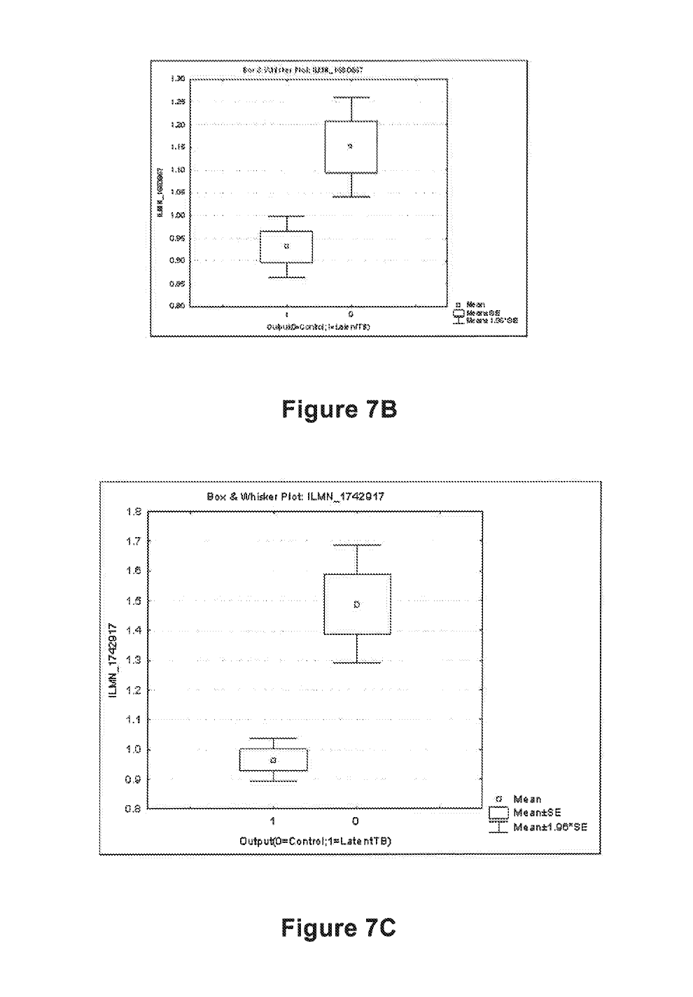

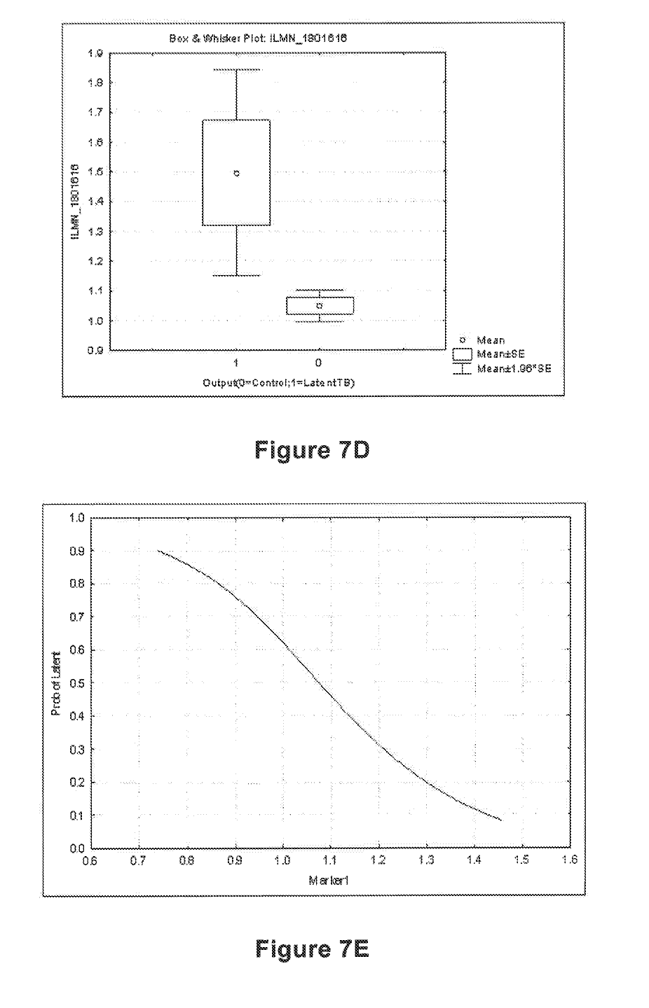

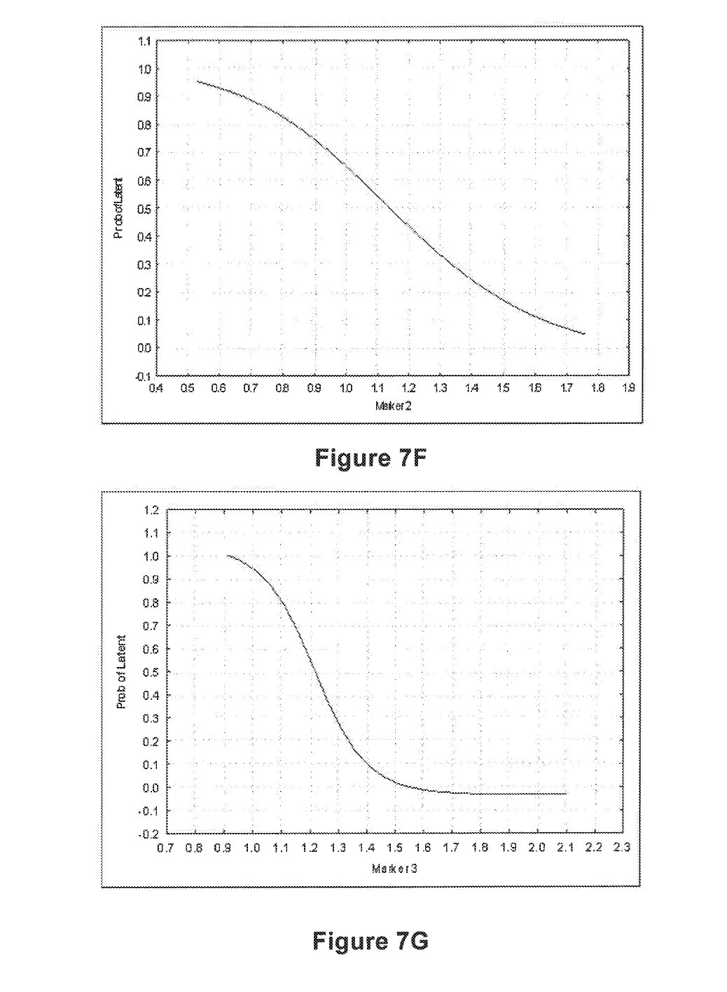

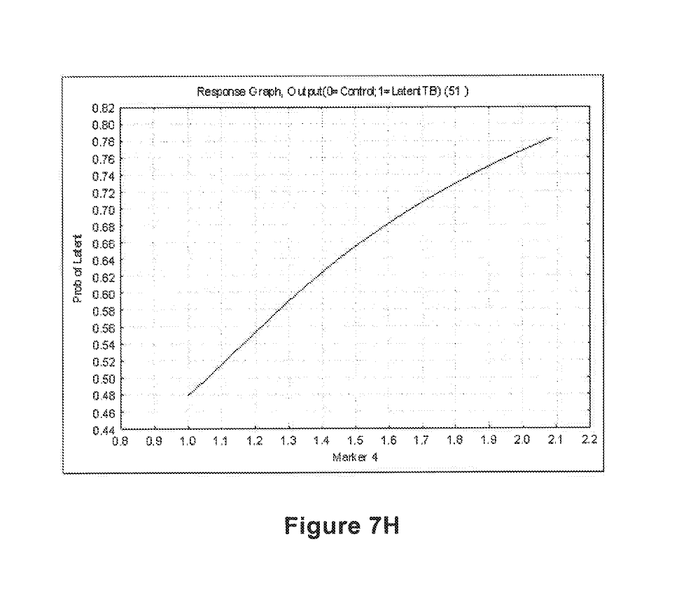

FIG. 7—shows a stepwise summary for the core set of biomarkers to distinguish between subjects that are not infected with M. tuberculosis and subjects with latent M. tuberculosis infection (NXNL1, PSMA7, C6orf61 and EMP1). Markers are added in a stepwise fashion to build an optimised panel for classification.

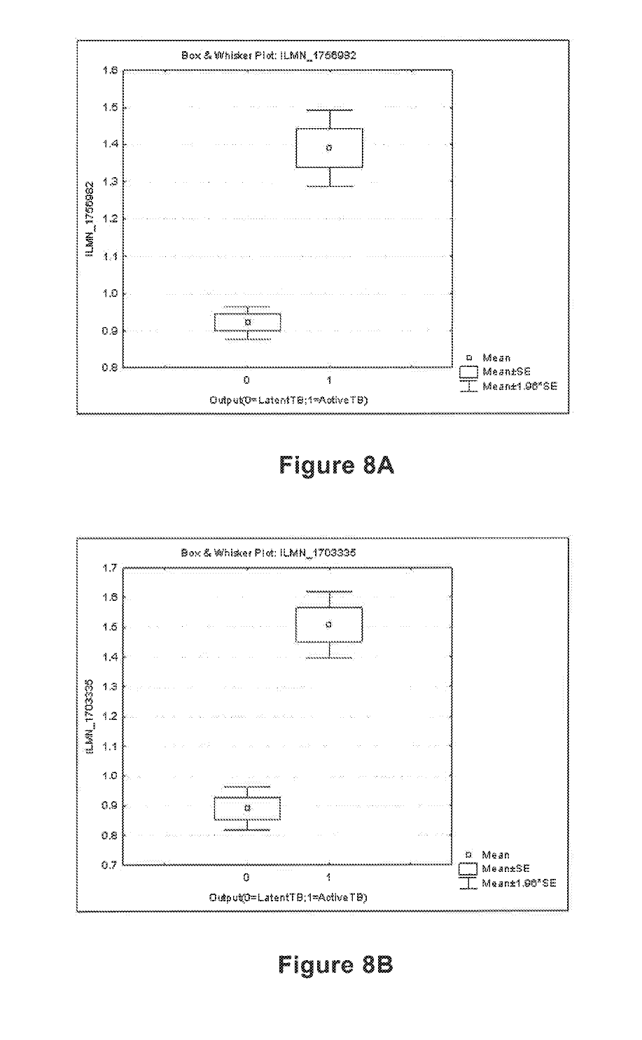

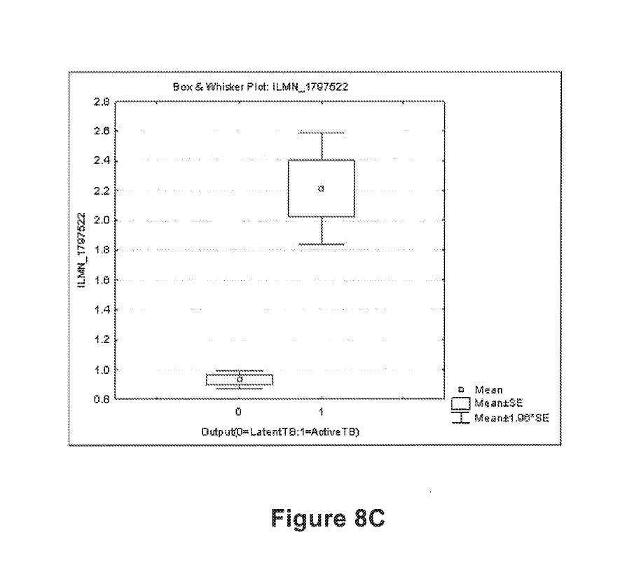

FIG. 8—shows a stepwise summary for the core set of biomarkers to distinguish between subjects that have active M. tuberculosis infection and subjects with latent M. tuberculosis infection (CLIC1, LACTB and DUSP3) Markers are added in a stepwise fashion to build an optimised panel for classification.

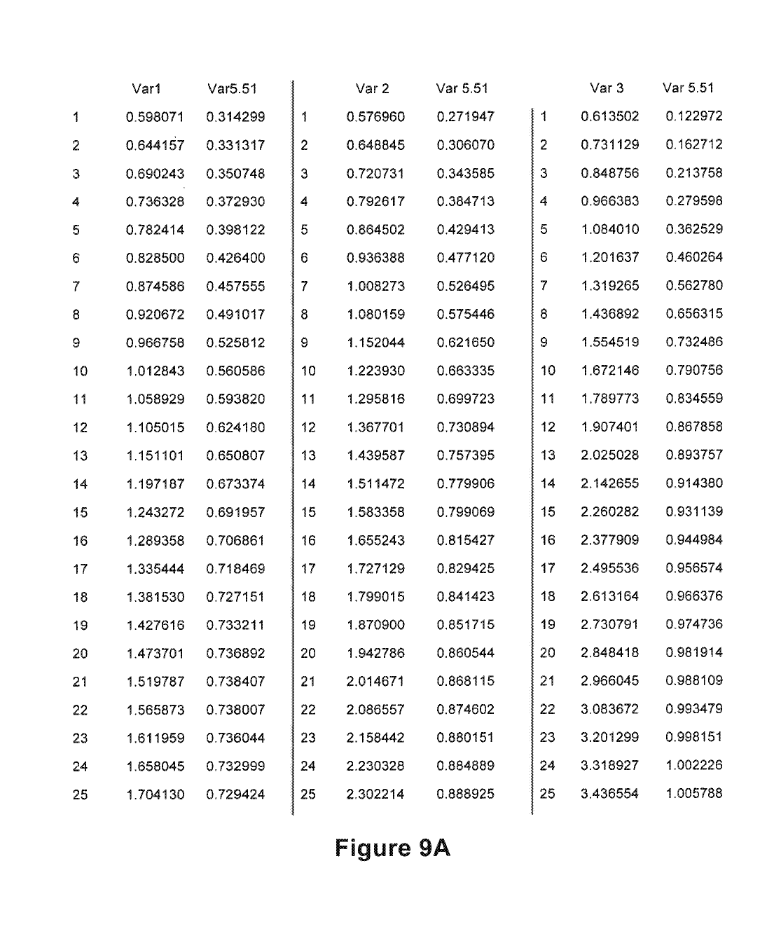

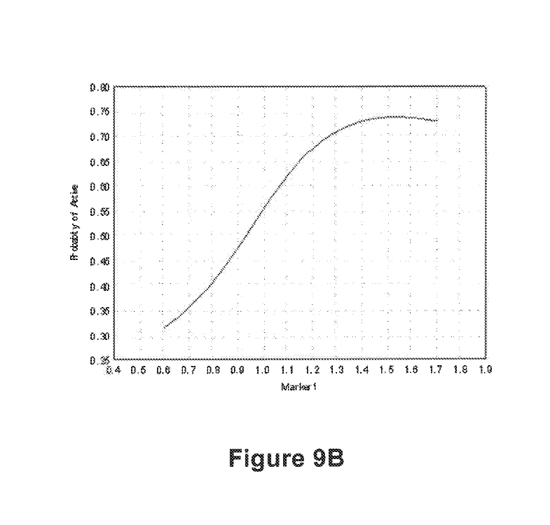

FIG. 9—shows response data and curve for the core set of biomarkers to distinguish between subjects that have active M. tuberculosis infection and subjects with latent M. tuberculosis infection (CLIC1, LACTB and DUSP3) relating the expression level of the marker to the probability of class membership.

Raw data used to produce gene panes was taken from the GENE EXPRESSION OMNIBUS database Code: E-GEOD-22098.

The data was analysed using a data mining algorithm and method both described in patent application number PCT/GB2009/051412 published as: WO2010046697 and claiming priority to GB 0819221.3. This method provided two panels of biomarkers.

1) A panel of biomarkers that are useful in differentiating between subjects that are not infected with M. tuberculosis and subjects with latent M. tuberculosis infection, these genes are: Homo sapiens nucleoredoxin-like 1 (NXNL1); Homo sapiens proteasome (prosome, macropain) subunit, alpha type, 7 (PSMA7); Homo sapiens chromosome 6 open reading frame 61 (C6orf61); and Homo sapiens epithelial membrane protein 1 (EMP1).
2. A panel of biomarkers that are useful in differentiating between subjects that have active M. tuberculosis infection and subjects with latent M. tuberculosis infection, these genes are: homo sapiens chloride intracellular channel 1 (CLIC1); Homo sapiens lactamase, beta (LACTB, nuclear gene encoding mitochondrial protein, transcript variant 1; and Homo sapiens dual specificity phosphatise 3 (vaccinia virus phosphatise VH1-related)(DUSP3).

Tables 1 shows the log of mean expression value for each gene in subjects 10 with latent M. tuberculosis infection and Control subjects not infected with M. tuberculosis and the p-value showing the statistical significance of the difference.

TABLE 1
Control v Latent

| | NXNL1 | PSMA7 | C6orf61 | EMP1 |
|---|---|---|---|---|
| Latent TB | 0.93 | 0.9 | 0.97 | 1.50 |
| Control | 1.15 | 1.29 | 1.49 | 1.05 |
| uninfected | | | | |
| P-value | 0.0016 | 0.0044 | 0.8 × 10⁻⁵ | 0.045 |

Tables 2 shows the log of mean expression value for each gene in subjects with latent M. tuberculosis infection and subjects with active M. tuberculosis infection and the p-value showing the statistical significance of the difference.

TABLE 2
Latent v Active

| | CLIC1 | LACTB | DUSP3 |
|---|---|---|---|
| Latent TB | 0.92 | 0.90 | 0.9 |
| Active TB | 1.40 | 1.5 | 2.2 |
| P-value | 0.4 × 10⁻⁵ | 0.14 × 10⁻⁵ | 0.1 × 10⁻⁶ |

TABLE 3
Control versus Latent probes of significance

| ILMN_Gene | Definition |
|---|---|
| LOC389541 | Homo sapiens similar to CG14977-PA |
| | (LOC389541), mRNA |
| MID1IP1 | Homo sapiens MID1 interacting protein |
| | 1 (gastrulation specific G12 homolog |
| | (zebrafish)) (MID1IP1), mRNA |
| KLRC3 | Homo sapiens killer cell lectin-like |
| | receptor subfamily C, member 3 |
| | (KLRC3), transcript variant 1, mRNA |
| KLF9 | Homo sapiens Kruppel-like factor 9 |
| | (KLF9), mRNA |
| GPR177 | Homo sapiens G protein-coupled |
| | receptor 177 (GPR177), transcript |
| | variant 1, mRNA |
| FBXO32 | Homo sapiens F-box protein 32 |
| | (FBXO32), transcript variant 2, mRNA. |
| TAZ | Homo sapiens tafazzin |
| | (cardiomyopathy, dilated 3A (X- |
| | linked); endocardial fibroelastosis 2; |
| | Barth syndrome) (TAZ), transcript |
| | variant 3, mRNA |
| C5ORF29 | Homo sapiens chromosome 5 open |
| | reading frame 29 (C5orf29), mRNA |
| HSDL1 | Homo sapiens hydroxysteroid |
| | dehydrogenase like 1 (HSDL1), mRNA. |
| CHUK | Homo sapiens conserved helix-loop- |
| | helix ubiquitous kinase (CHUK), |
| | mRNA |
| LOC652062 | PREDICTED: Homo sapiens similar to |
| | Mitochondrial carnitine/acylcarnitine |
| | carrier protein (Carnitine/acylcarnitine |
| | translocase) (CAC) (LOC652062), |
| | mRNA |
| HIP1 | Homo sapiens huntingtin interacting |
| | protein 1 (HIP1), mRNA |
| C6ORF60 | Homo sapiens chromosome 6 open |
| | reading frame 60 (C6orf60), transcript |
| | variant 1, mRNA |
| MTMR11 | Homo sapiens myotubularin related |
| | protein 11 (MTMR11), transcript |
| | variant 1, mRNA. |

TABLE 4
Active versus Latent probes of significance

| ILMN_Gene | Definition |
|---|---|
| LACTB | Homo sapiens lactamase, beta |
| | (LACTB), nuclear gene encoding |
| | mitochondrial protein, transcript |
| | variant 1, mRNA. |
| SRBD1 | Homo sapiens S1 RNA binding domain |
| | 1 (SRBD1), mRNA. |
| DUSP3 | Homo sapiens dual specificity |
| | phosphatase 3 (vaccinia virus phosphatase |
| | VH1-related) (DUSP3), mRNA. |
| ATG3 | Homo sapiens ATG3 autophagy related |
| | 3 homolog (S. cerevisiae) (ATG3), |
| | mRNA. |
| JAK2 | Homo sapiens Janus kinase 2 (a protein |
| | tyrosine kinase) (JAK2), mRNA. |
| PSMB8 | Homo sapiens proteasome (prosome, |
| | macropain) subunit, beta type, 8 (large |
| | multifunctional peptidase 7) (PSMB8), |
| | transcript variant 2, mRNA. |
| PSME1 | Homo sapiens proteasome (prosome, |
| | macropain) activator subunit 1 (PA28 |
| | alpha) (PSME1), transcript variant 1, |
| | mRNA. |
| ACOT9 | Homo sapiens acyl-CoA thioesterase 9 |
| | (ACOT9), transcript variant 2, mRNA. |
| IFI30 | Homo sapiens interferon, gamma- |
| | inducible protein 30 (IFI30), mRNA. |
| SORT1 | Homo sapiens sortilin 1 (SORT1), |
| | mRNA. |
| GSR | Homo sapiens glutathione reductase |
| | (GSR), mRNA. |
| TAP1 | Homo sapiens transporter 1, ATP- |
| | binding cassette, sub-family B |
| | (MDR/TAP) (TAP1), mRNA. |
| GRN | Homo sapiens granulin (GRN), mRNA. |
| IRF1 | Homo sapiens interferon regulatory |
| | factor 1 (IRF1), mRNA. |

### Sequences Used in the Application

TABLE 5
gene names and SEQ ID NO

| ILMN Gene | | |
|---|---|---|
| (names | | |
| shown | SEQ | |
| in FIGS. | NO | Sequence used to |
| 3 and 4) | ID | identify the gene |
| NXNL1 | 1 | GAGACCCTGACTCTACGAAAATTAAAAGTT |
| | | AGCCCGGTGTGGTGGCGCGC |
| PSMA7 | 2 | CGCTTGCATGCTCACCTCTGGCAGCAGGGC |
| | | AGTCACGGCTCCGCCATGGA |
| C6ORF61 | 3 | GGCAGGATGATTGCTTAAGCCCAGGAGTTC |
| | | GGGGTTACAGTGAGCTGTGG |
| EMP1 | 4 | GAGGGCAAGCCACCAAATTACCTAGGCTGA |
| | | GGTTAGAGAGATTGGCCAGC |
| CLIC1 | 5 | GAGCTCGCCTATGAGCAAGTGGCAAAGGCC |
| | | CTCAAATAAGCCCCTCCTGG |
| LACTB | 6 | ATACTGGAGGGGCAGTGGGTGCCAGTAGTG |
| | | TCCTGCTGGTCCTTCCTGAA |
| DUSP3 | 7 | CCATGGTGATGGATGGTTTGGAAAGGGAAT |
| | | GTTGGTGCCTTTTGTGCCAC |
| LOC389541 | 8 | TGTGGTGAAGAGGCAGAACCGAGGTCGGG |
| | | AGCCCATTGATGTCTGAGCCT |
| MID1IP1 | 9 | CCCCAGTGTGTATAAGCTGGCATTTCGCCA |
| | | GCTTGTACGTAGCTTGCCAC |
| KLRC3 | 10 | GGGAAGAGAGTTTGCAGGCCTGTGCTTCAA |
| | | AGAACTCTTCTAGTCTGCTT |
| KLF9 | 11 | GCCCTTCACCATTGTGGAATGATGCCCTGG |
| | | CTTTAAGGTTTAGCTCCACA |
| GPR177 | 12 | TGAGATCTACAAGTTGACCCGCAAGGAGGC |
| | | CCAGGAGTAGGAGGCTGCAG |
| FBXO32 | 13 | GAAGGGTCCCCCTGCTGACTGGAGAGCTGG |
| | | GAATATGGCATTTGGACACT |
| TAZ | 14 | GACAGATTTGTTCATAGACCCTCTCAAGTG |
| | | CCCTCTCCGAGCTGGTAGGC |
| C5ORF29 | 15 | GCCTCTTCTCTCAAGCCTGCTTCAGATCATA |
| | | AGTTCTTCCACACATCTCC |
| HSDL1 | 16 | GTGCCGAGCTGTCCATAGCTGCAGTGAAAG |
| | | GTGAAGAGCAAGACCTTCTC |
| C6ORF60 | 17 | GCTTCTCCAGATCCCCAGCGCCAGGAGTGG |
| | | TTTGCCCGGTACTTCACATT |
| CHUK | 18 | CCTTTATTTTGCTGCTTGATGATGAGAGGG |
| | | AGGGCTGCTGCCACAGACTG |
| LOC652062 | 19 | ATGGAGCTGTCTTTCAGATCTTTCCTGGGAT |
| | | TACCCCTGCCTACCCCCAG |
| HIP1 | 20 | TGCAGCCGTCCATAGCAGTACCCCTAAAAT |
| | | CCCACCAGAATACGGGTCCC |
| MTMR11 | 21 | CACCAGAAGTAGCAGAGAAGCAGGGGGCC |
| | | AGAGCTACAACAGTATTCTTC |
| SRBD1 | 22 | ACTTCTACTTGCCAACATCTGCCTTGCTGGA |
| | | CTTGTATGGGATTGTCTCC |
| ATG3 | 23 | AGTGACCATTGAAAATCACCCTCATCTGCC |
| | | ACCACCTCCCATGTGTTCAG |
| JAK2 | 24 | TTGTCATCCTTTGAGCTGCTGACTGCCAATA |
| | | ACATTCTTCGATCTCTGGG |
| PSMB8 | 25 | GTCGCTCGGACCCAGGACACTACAGTTTCT |
| | | CTATGCGATCTCCAGAGCTC |
| PSME1 | 26 | ACCGGGACATCCGGCTGATGGTCATGGAGA |
| | | TCCGCAATGCTTATGCTGTG |
| ACOT9 | 27 | GGACATTAAGTTCAGTGGCCATGTTAGCTG |
| | | GGTCGGGAAGACATCCATGG |
| IFI30 | 28 | TGGAAGATCAGACCCAGCTCCTTACCCTTG |
| | | TCTGCCAGTTGTACCAGGGC |
| LACTB | 29 | ATACTGGAGGGGCAGTGGGTGCCAGTAGTG |
| | | TCCTGCTGGTCCTTCCTGAA |
| DUSP3 | 30 | CCATGGTGATGGATGGTTTGGAAAGGGAAT |
| | | GTTGGTGCCTTTTGTGCCAC |
| SORT1 | 31 | GGTCCCCATGTGCCTGTTGTTCAGCCCTCTC |
| | | TCTTGTTCCCTTTCTGAGC |
| GSR | 32 | GAACCAGGAGACACGTGTGGCGGGCAGTG |
| | | GGACCCATAGATCTTCTGAAA |
| TAP1 | 33 | GTAACGGAGTTTAGAGCCAGGGCTGATGCT |
| | | TTGGTGTGGCCAGCACTCTG |
| GRN | 34 | AAGGCTCGATCCTGCGAGAAGGAAGTGGTC |
| | | TCTGCCCAGCCTGCCACCTT |
| IRF1 | 35 | CCTCAACAGGCCCAGGGAGGGAAGTGTGA |
| | | GCGCCTTGGTATGACTTAAAA |

1-24. (canceled)

25. A method of determining the M. tuberculosis infection status of a subject comprising:
(a) providing a sample of material obtained from the subject; and
(b) determining the expression level of one or more genes selected from the group consisting of: NXNL1, PSMA7, C6orf61, EMP1, CLIC1, LACTB and DUSP3 in the sample.

26. The method of claim 25, further comprising the step of:
(c) determining the expression level of one or more genes listed in table 3 or table 4.

27. The method of claim 25, further comprising:
employing the expression level determined in (b); and
optionally determining the expression level of one or more genes listed in table 3 or table 4;
to distinguish between subjects not infected with M. tuberculosis, subjects with latent M. tuberculosis infection and subjects with active M. tuberculosis infection.

28. The method of claim 25, for use in one or more of the following: diagnosing whether or not a subject has M. tuberculosis infection; advising on the prognosis for a subject with a M. tuberculosis infection; and monitoring the effectiveness or response of a subject to a particular treatment for infection by M. tuberculosis.

29. The method of claim 25, wherein altered expression level of one or more genes selected from: NXNL1, PSMA7, C6orf61 and EMP1 can differentiate between subjects not infected with M. tuberculosis and subjects with latent M. tuberculosis infection.

30. The method of claim 25, wherein decreased expression level of the genes NXNL1, PSMA7 and C6orf61 and increased expression level of the gene EMP1 indicates that a subject has latent M. tuberculosis infection.

31. The method of claim 25, wherein altered expression level of one or more genes selected from NXNL1, PSMA7, C6orf61, EMP1 and at least one gene selected from table 3 can differentiate between subjects not infected with M. tuberculosis and subjects with latent M. tuberculosis infection.

32. The method of claim 25, wherein altered expression level of one or more genes selected from NXNL1, PSMA7, C6orf61, EMP1 and one, two or three genes selected from table 3 can differentiate between subjects not infected with M. tuberculosis and subjects with latent M. tuberculosis infection.

33. The method of claim 25, wherein decreased expression level of the genes NXNL1, PSMA7 and C6orf61 and increased expression level of the gene EMP1 and altered expression level of one, two or three genes selected from table 3 indicates that a subject has latent M. tuberculosis infection.

34. The method according to claim 33, wherein increased expression of LOC389541 and/or increased expression of MID1IP1 and/or increased expression of KLRC3 and/or increased expression of KLF9 and/or decreased expression GPR117 and/or increased expression of FBXO32 and/or decreased expression of TAZ and/or increased expression of C5ORF29 and/or decreased expression of HSDL1 and/or increased expression of CHUK and/or increased expression of LOC652062 and/or decreased expression of HIP1 and/or increased expression of C6ORF60 and/or increased expression of MTMR11 indicates that a subject has latent M. tuberculosis infection.

35. The method of claim 25, wherein altered expression level of one or more genes selected from: CLIC1, LACTB and DUSP3 can differentiate between subjects with latent M. tuberculosis infection and subjects with active M. tuberculosis infection.

36. The method of claim 25, wherein altered expression level of all of the genes CLIC1, LACTB and DUSP3 can differentiate between subjects with latent M. tuberculosis infection and subjects with active M. tuberculosis infection.

37. The method of claim 25, wherein increased expression level of all of the genes CLIC1, LACTB and DUSP3 indicates a subject with active M. tuberculosis infection.

38. The method of claim 25, wherein increased expression level of all of the genes CLIC1, LACTB and DUSP3 and increased expression level of one, two or three genes selected from table 4 indicates a subject with active M. tuberculosis infection.

39. The method of claim 25, wherein the altered expression level is an expression level that is higher or lower than the expression level expected in a subject not infected with M. tuberculosis.

40. The method of claim 25, wherein the expression level of the gene is determined using the level of mRNA encoded by the selected gene present in the sample.

41. The method of claim 25, wherein the expression level of the gene is determined using quantitative PCR.

42. The method of claim 25, wherein the expression level of one or more further genes is also determined.

43. A kit for use in determining the M. tuberculosis infection status in a subject comprising at least one agent for determining the expression level of one or more genes selected from the group consisting of: NXNL1, PSMA7, C6orf61, EMP1, CLIC1, LACTB and DUSP3 in a sample from a subject and instructions for determining the M. tuberculosis infection status of the subject.

44. The kit of claim 43, wherein the agent is an oligonucleotide.

45. An oligonucleotide capable of detecting the presence or expression level of a gene expression product from a gene selected from the group consisting of NXNL1, PSMA7, C6orf61, EMP1, CLIC1, LACTB and DUSP3 in a sample from a subject.
